(19) United States
(12) Patent Application Publication
(10) Pub. No.: US 2022/0409831 A1
(43) Pub. Date: Dec. 29, 2022

(54) INHALER DEVICES, MEDICATION FORMULATIONS USED THEREWITH AND METHODS OF MANUFACTURE

(71) Applicant: Genentech, Inc., South San Francisco, CA (US)

(72) Inventors: Avi Eliahu, South San Francisco, CA (US); Stuart Abercrombie, Cambridge (GB)

(21) Appl. No.: 17/806,252

(22) Filed: Jun. 9, 2022

Related U.S. Application Data
(63) Continuation of application No. PCT/US2020/063889, filed on Dec. 9, 2020.
(60) Provisional application No. 62/945,748, filed on Dec. 9, 2019.

(51) Int. Cl. A61M 15/00

(52) U.S. Cl. CPC A61M 15/0035; A61M 15/0008; A61M 15/0021

(57) ABSTRACT

Exemplary dry powder inhaler devices may include a housing body having a medicine capsule recess therein, a pair of air inlets each fluidically connecting to the recess, and an outlet body coupled to the housing body. Each inlet may have a width no greater than 1.17 mm. The outlet body may have a channel fluidically connecting the recess to an opening and configured to allow a user to draw air through the opening, thereby allowing an airstream to be drawn through the air inlets, into the housing body recess where it causes the capsule to spin and eject its contents into the airstream, and through the outlet body channel and opening to deliver the substance into the user's lungs. Dry powder respirable drug blend formulations, methods of manufacturing the formulations and drug/device combinations are also provided.

### CROSS REFERENCE TO RELATED APPLICATIONS

This application is a continuation of International Application No. PCT/US2020/063889, filed Dec. 9, 2020, which claims the benefit of priority to U.S. Application No. 62/945,748 filed Dec. 9, 2019 and is herein incorporated by reference in its entirety.

### INCORPORATION BY REFERENCE

All publications and patent applications mentioned in this specification are herein incorporated by reference for all intents and purposes to the same extent as if each individual publication or patent application was specifically and individually indicated to be incorporated by reference.

### FIELD

Embodiments of the disclosure relate generally to inhaler devices. Specifically, some implementations of the present disclosure relate to dry powder inhaler devices, dry powder respirable drug blend formulations, methods of manufacturing the formulations and drug/device combinations.

### BACKGROUND

The present disclosure relates to inhaler devices, such as for inhaling dry powder medications to treat asthma. Inhaler devices for inhaling the contents of a capsule for medical uses are already known. Available inhalers, however, are not fully satisfactory from an operating standpoint and are susceptible to improvements.

U.S. Pat. No. 7,284,552 to Mauro Citterio, issued on Oct. 23, 2007 and entitled INHALER DEVICE, provides an example of a prior art inhaler device similar to those provided herein. The inhaler device includes an inhaler body defining a recess for a medicine capsule holding a substance to be inhaled, and a nosepiece/mouthpiece communicating with the capsule recess. The device also includes at least one perforating element coupled to the inhaler body and provided for perforating the capsule for allowing an outside airflow to be mixed with the capsule contents and inhaled through the nosepiece/mouthpiece.

U.S. Pat. No. 8,479,730 to Dominik Ziegler et al., issued on Jul. 9, 2013 and entitled INHALER DEVICE, provides another example of a prior art inhaler device. The inhaler device of the 8,479,730 patent is similar in construction and operation to that of the 7,284,552 patent, but has a mouthpiece that is pivotally attached to an edge of the inhaler body.

What is needed and not provided by prior art inhalers and drug formulations are products that deliver drug doses more effectively and repeatably.

### SUMMARY

According to aspects of the disclosure, improved dry powder inhaler devices are provided. Dry powder respirable drug blend formulations, methods of manufacturing the formulations and drug/device combinations are also provided.

In some embodiments, a suction operated inhaler device includes a bottom inhaler body and a top mouthpiece. In these embodiments, the bottom inhaler body has an air inlet hole and further defines a recess configured to hold therein a capsule containing a substance to be inhaled. The top mouthpiece communicates with the recess and has a bottom flange that is rotatably coupled to the bottom inhaler body. At least two operating conditions are provided as the top mouthpiece is manually rotated by an inhaler device user. The two operating conditions include an open condition in which the recess for the capsule can be accessed to engage therein a new capsule or to withdraw therefrom a used capsule, and a closed use condition in which the inhaler device mouthpiece can be operated. The inhaler device further includes at least one perforating needle associated with the inhaler body. The at least one perforating needle is adapted to perforate the capsule to allow a contents of the capsule to enter the capsule recess. This allows an inhaling suction generated airflow passing through a first air inlet hole to mix with the contents of the capsule for inhaling the contents in the recess through the mouthpiece. In these embodiments, the first air inlet hole has a width no greater than 1.17 mm.

In some embodiments, a dry powder inhaler device includes a housing body, a pair of air inlets and an outlet body. In these embodiments, the housing body has a cylindrically shaped recess therein. The recess has a longitudinal axis, a height along the longitudinal axis that is larger than a diameter of a capsule containing a substance to be inhaled, and a diameter transverse to the longitudinal axis that is larger than a length of the capsule. This arrangement allows the capsule room to spin within the recess generally about a transverse axis of the capsule and generally about the longitudinal axis of the recess. The pair of air inlets each fluidically connect the recess to an aperture on an exterior surface of the housing body. Each inlet has a surface that is aligned with a tangent to an outer surface of the recess. Each inlet has a height no greater than the height of the recess and a width no greater than 1.17 mm. The outlet body is coupled to the housing body and has a channel fluidically connecting the recess to an opening. This arrangement is configured to allow a user to draw air through the opening, thereby allowing an airstream to be drawn through the air inlets, into the housing body recess where it causes the capsule to spin and eject its contents into the airstream. The airstream is further drawn through the outlet body channel and through the opening to deliver the substance into the user's lungs.

In some embodiments, a suction operated inhaler device includes a bottom inhaler body and a top mouthpiece. In these embodiments, the bottom inhaler body has an air inlet hole and further defines a recess configured to hold therein a capsule containing a substance to be inhaled. The top mouthpiece communicates with the recess and has a bottom flange that is rotatably coupled to the bottom inhaler body. At least two operating conditions are provided as the top mouthpiece is manually rotated by an inhaler device user. The two operating conditions include an open condition in which the recess for the capsule can be accessed to engage therein a new capsule or to withdraw therefrom a used capsule, and a closed use condition in which the inhaler device mouthpiece can be operated. The inhaler device further includes at least one perforating needle associated with the inhaler body. The at least one perforating needle is adapted to perforate the capsule to allow a contents of the capsule to enter the capsule recess. This allows an inhaling suction generated airflow passing through a first air inlet hole to mix with the contents of the capsule for inhaling the contents in the recess through the mouthpiece. In these embodiments, the first air inlet hole has a width no greater than 1.17 mm. The first air inlet hole and the second air inlet hole each have a constant, rectangular, transverse cross-section along a predetermined length of the air inlet hole. In these embodiments, the predetermined length is about 4.50 mm, the height of each rectangular cross-section is about 5.50 mm and the width of each rectangular cross-section is between about 1.02 mm and about 1.12 mm, inclusive. The first air inlet hole has an outwardly facing radius of about 1.60 mm. The capsule recess has an outer wall with a constant diameter of about 19.00 mm, the outer wall being continuous with no air pockets therein. The mouthpiece has an inside diameter of about 11.00 mm. The inhaler device has an internal bypass gap located between the bottom flange of the top mouthpiece and the bottom inhaler body, the internal bypass gap being no greater than about 0.1 mm. In these embodiments, the device has an airflow resistance of about 0.128 cmH₂O^{0.5}/LPM, which is equivalent to a flow rate of 50LPM at 4 kPa.

In some embodiments, a dry powder respirable drug blend formulation includes a lactose excipient and a small molecule drug manufactured to treat asthma. The drug may include micronized crystal particles having a median size of 2 to 4 microns. In these embodiments, a percentage of drug weight in the formulation is more than 10% and less than 70%.

In some embodiments, a method of manufacturing a dry powder respirable drug blend formulation includes providing a small molecule drug and a lactose excipient. The small molecule drug is manufactured to treat asthma and includes micronized crystal particles having a median size of 2 to 4 microns. The drug is blended with the lactose such that a percentage of drug weight in a final blend is more than 10% and less than 70%.

In some embodiments, an asthma treatment product includes a dry powder inhaler device and at least one medicine capsule. The inhaler device is configured to receive the at least one medicine capsule which contains a dry powder respirable drug blend formulation. In these embodiments, the dry powder inhaler device includes a housing body, a pair of air inlets and an outlet body. The housing body has a cylindrically shaped recess therein. The recess has a longitudinal axis, a height along the longitudinal axis that is larger than a diameter of a capsule containing a substance to be inhaled, and a diameter transverse to the longitudinal axis that is larger than a length of the capsule. This arrangement allows the capsule room to spin within the recess generally about a transverse axis of the capsule and generally about the longitudinal axis of the recess. The pair of air inlets each fluidically connect the recess to an aperture on an exterior surface of the housing body. Each inlet has a surface that is aligned with a tangent to an outer surface of the recess. Each inlet has a height no greater than the height of the recess and a width no greater than 1.17 mm. The outlet body is coupled to the housing body and has a channel fluidically connecting the recess to an opening. This arrangement is configured to allow a user to draw air through the opening, thereby allowing an airstream to be drawn through the air inlets, into the housing body recess where it causes the capsule to spin and eject its contents into the airstream. The airstream is further drawn through the outlet body channel and through the opening to deliver the substance into the user's lungs. In these embodiments, the dry powder respirable drug blend formulation includes a lactose excipient and a small molecule drug manufactured to treat asthma. The drug includes micronized crystal particles having a median size of 2 to 4 microns. In these embodiments, the percentage of drug weight in the formulation is more than 10% and less than 70%.

### BRIEF DESCRIPTION OF THE DRAWINGS

A better understanding of the features and advantages of the present disclosure will be obtained by reference to the following detailed description that sets forth illustrative embodiments, in which the principles of the disclosure are utilized, and the accompanying drawings of which:

FIG. 1 is an exploded perspective view of an exemplary embodiment of an inhaler device according to the present disclosure;

FIG. 2 is a further perspective view of the exemplary inhaler device shown in an open condition thereof, i.e. in the capsule loading position thereof;

FIG. 3 is a view similar to FIG. 2, but illustrating the inhaler device according to the present disclosure during the use thereof;

FIG. 4 is an elevation cross-sectional view of the inhaler device, shown with a capsule arranged therein, but in a non-perforated condition;

FIG. 5 is a view similar to FIG. 4, but illustrating the inhaler device according to the present disclosure during the capsule perforating operation;

FIG. 6 is a top plan view, as partially cross-sectioned, of the inhaler device according to the present disclosure;

FIG. 7 is a perspective cross-sectional view of the inhaler device illustrating airflow through the device;

FIG. 8 is a top plan view of the inhaler device illustrating features of the inlets;

FIG. 9 is a partial view shown by the circular region in FIG. 8;

FIG. 10 is a graph showing the relationship between airflow resistance and emitted dose in a prior art inhaler device;

FIG. 11 is a top plan view of the mouthpiece grid of four different inhaler devices depicting variations in mouthpiece ID and grid open area;

FIG. 12 is a diagram that schematically illustrates a first method of formulating a dry powder respirable drug blend having a 70% drug load according to aspects of the present disclosure;

FIG. 13 is a diagram that schematically illustrates a second method of formulating a dry powder respirable drug blend having a 50% drug load according to aspects of the present disclosure;

FIG. 14 is a diagram that schematically illustrates a third method of formulating a dry powder respirable drug blend having a 50% drug load according to aspects of the present disclosure;

FIG. 15 is a table that summarizes six formulations manufactured using the methods shown in FIGS. 12-14;

FIG. 16 is a table that shows results of blend content uniformity (BCU) testing performed on the six formulations summarized in FIG. 15;

FIG. 17 is a table that shows capsule filling data for the four passing formulations shown in FIGS. 15 and 16;

FIG. 18 is a table that shows capsule content uniformity data for the four formulations shown in FIG. 17;

FIG. 19 is a table that shows emitted dose results for the four formulations above, using both a prior art inhaler device and an inhaler device constructed according to aspects of the present disclosure;

FIG. 20 is a table that shows aerodynamic particle size distribution (APSD) test results for the four formulations above, using both a prior art inhaler device and an inhaler device constructed according to aspects of the present disclosure;

FIG. 21 is a graph that shows the APSD profile for the four formulations above using a prior art inhaler device;

FIG. 22 is a graph that shows the APSD profile for the -003 formulation using both a prior art inhaler device and an inhaler device constructed according to aspects of the present disclosure;

FIG. 23 is a graph that shows the APSD profile for the -004 formulation using both a prior art inhaler device and an inhaler device constructed according to aspects of the present disclosure;

FIG. 24 is a graph that shows the APSD profile for the -006 formulation using both a prior art inhaler device and an inhaler device constructed according to aspects of the present disclosure;

FIG. 25 is a graph that shows the APSD profile for the -007 formulation using both a prior art inhaler device and an inhaler device constructed according to aspects of the present disclosure;

FIG. 26 is a table that shows APSD data for the -004 formulation after being placed under set environmental conditions;

FIG. 27 is a table that shows APSD data for the -007 formulation after being placed under set environmental conditions;

FIG. 28 is a table that shows emitted dose data for the -004 and -007 formulations after being placed under set environmental conditions.

### DETAILED DESCRIPTION

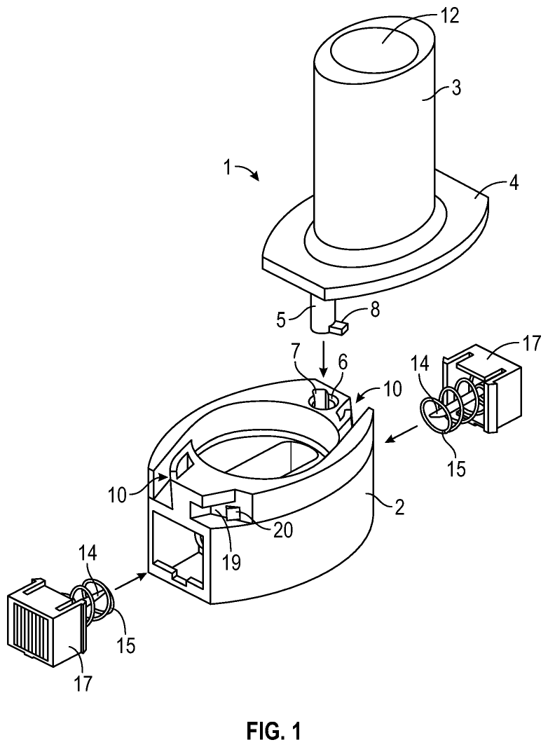

Referring to the reference numerals of the above-mentioned figures, an exemplary inhaler device 1 constructed according to aspects of the present disclosure is described below. As best seen in FIG. 1, the exemplary inhaler device 1 comprises an inhaler mouthpiece 3, including a flange 4, having a peg 5 which can be engaged in a corresponding hole 6 formed in an inhaler body 2. While the term “mouthpiece” is used herein, it is to be understood that in some embodiments this feature may be used as a mouthpiece and or a nosepiece.

The hole 6 is provided with a longitudinal slot (not shown), that can engage a cross tooth 8 of the peg 5, and a bottom ring-like recess, not specifically shown, in which the tooth 8 can slide.

Thus, it is possible to engage the peg 5 in the hole, by causing the tooth 8 to pass through the slot 7 and, upon achieving the bottom, it is possible to fully rotate the peg 5 in its hole 6, thereby also rotating the inhaler mouthpiece 3 with respect to the inhaler body 2.

The inhaler mouthpiece 3 can be locked in its closed condition, shown in FIGS. 3-6, by a snap type of locking means, including a hook portion 18 of the flange 4 having a small ridge, not shown, for engaging a corresponding ridge 20 formed inside a latching recess 19, defined in the inhaler body 2.

The inhaler body 2 is moreover provided with a recess for the capsule, the recess being upward opened and communicating with the outside through a perforated plate or grid 11, included in the inhaler mouthpiece 3 at the flange 4 and designed for separating the capsule recess 9 from the duct 12 of the mouthpiece.

A capsule 13 can be engaged in the recess 9, the capsule being of a per se known type and adapted to be perforated to allow the drug contents held therein to be easily accessed, the perforating operation being performed by any suitable perforating means.

In the disclosed embodiment, the perforating means comprise a pair of perforating needles 14 which can transversely slide as counter-urged by resilient elements comprising, in this embodiment, coil springs 15; each coil spring coaxially encompassing the perforating needle 14 and operating between a respective abutment element 16, rigid with the inhaler body 2, and a hollow push-button element 17. The perforating needles 14 may be similar to hollow hypodermic needles and have a single-side beveled tip, for facilitating the perforating needles 14 in perforating the coating of the capsule 13. In other implementations, the perforating needles 14 may be solid and or have other tip configurations.

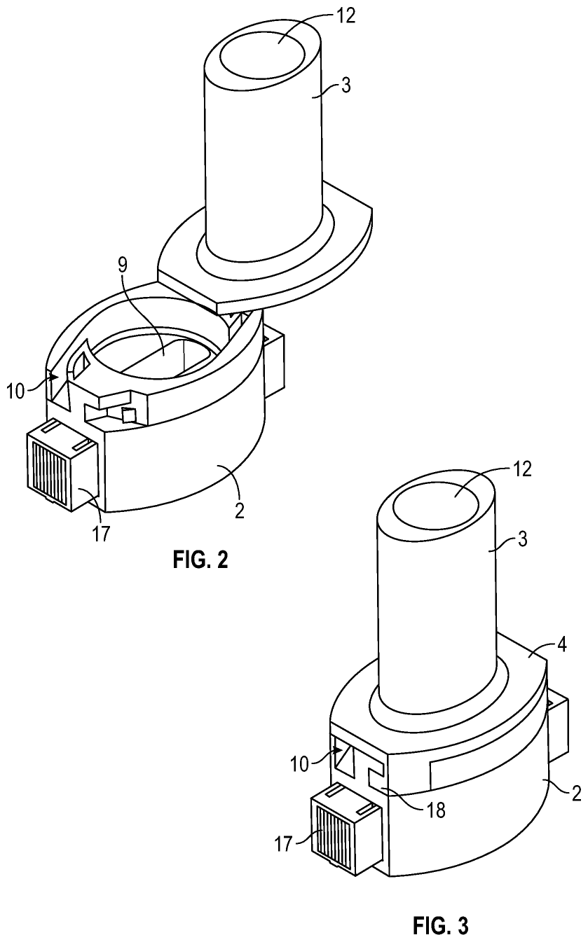

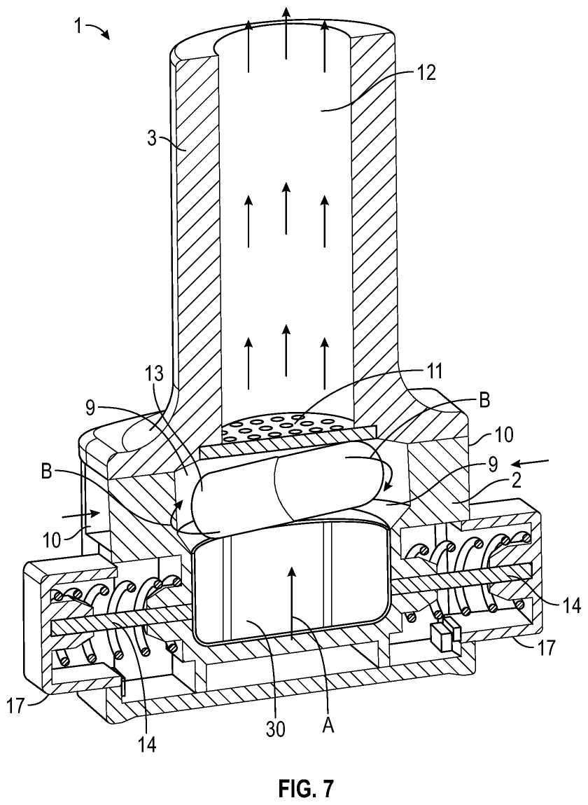

The operation of the inhaler device according to the present disclosure is as follows. In the open condition, as shown in FIG. 2, a capsule is engaged in the capsule recess 9 and the mouthpiece 3 is snapped closed on the inhaler body 2. By pressing the push-button elements 17, the perforating needles 14 will perforate the capsule 13, thereby its contents, usually a fine powder, will be communicated with the capsule recess. By applying suction on the mouthpiece 3, an airflow is generated which, coming from the outside through the inlets 10, will enter the capsule recess, thereby mixing with the capsule contents. The tangential orientation of inlets 10 relative to the capsule recess 9 causes the incoming air to generate a swirling airflow. This swirling airflow lifts capsule 13 upward (shown by arrow A in FIG. 7) out of capsule pocket 30 and into the larger, upper portion of capsule recess 9. The swirling airflow further spins capsule 13 within recess 9 as shown by arrows B, generally about a transverse axis of the capsule and generally about the longitudinal axis of recess 9 (i.e. a generally vertical axis in FIG. 7). However, since the diameter of recess 9 is larger than the length of capsule 13, the capsule may travel around recess 9 as it spins rather than spinning around a single fixed axis. Centrifugal force from the spinning capsule 13 assists its contents in exiting the pierced ends of the capsule, where it is aerosolized by the swirling airflow, passes through mouthpiece grid 11 and duct 12, and is inhaled by the user. In some embodiments, dry powder deagglomeration is achieved by: 1) shear through the pierced holes in the capsule; 2) turbulence from swirling airflow in the capsule chamber; and 3) particle collisions (with the walls of the device, with the mouthpiece grid, and with other particles.)

Inhaler device 1 has a very simple construction. A further advantage of inhaler device 1 is the specifically designed configuration of the perforating needles that can be assimilated, as stated, to hypodermic needles. Since this type of needle presents a very small resistance against perforation and a very accurate operation, it is possible to use needles having a comparatively large diameter, without damaging the capsule, thereby providing a very simple perforating operation. The use of a small number of perforating needles, only two in some embodiments, allows reducing the contact surface between the needle and capsule (the perforated cross section being the same), with a consequent reduction of friction and of the problems affecting the prior inhalers.

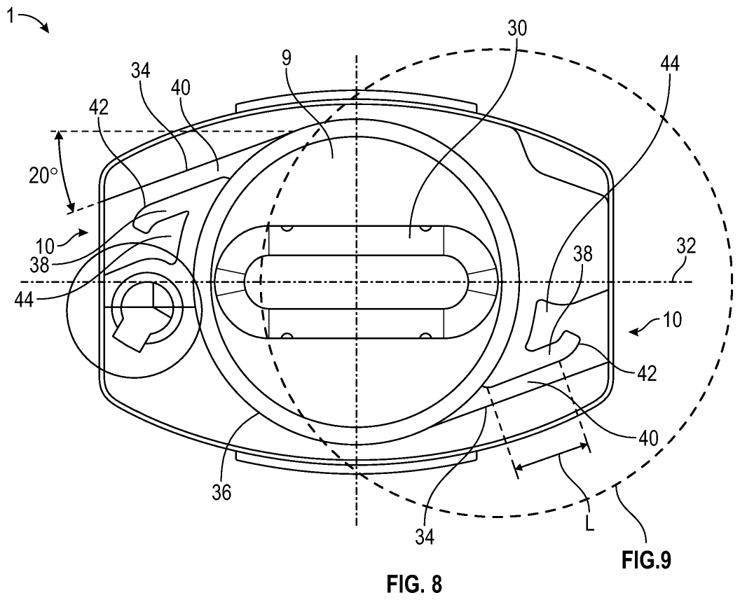

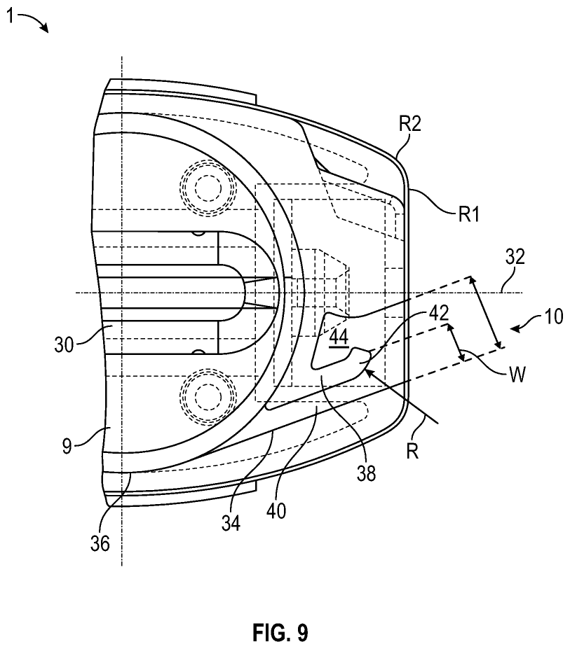

Referring to FIGS. 8 and 9, further details of air inlets 10 according to aspects of the present disclosure are provided. As best seen in FIG. 8, exemplary inhaler device 1 is provided with two air inlets 10 located on opposite sides of device 1. Each inlet 10 is oriented at a 20 degree angle relative to a longitudinal centerline 32 of device 10, as shown. Each inlet 10 also has an outer surface 34 that is aligned with a tangent to an outer surface 36 of the capsule recess 9. Each inlet 10 may be provided with a divider 38 that, together with outer surface 34, defines an inner inlet channel 40 that is necked down and shorter than the entire inlet 10. Each divider 38 may be provided with a curved portion 42 on its distal, outwardly facing end as shown. Each divider 38 also has a length L as shown, which does not include the curved portion 42. In some embodiments, length L of inlet dividers 38 is about 4.50 mm and defines a necked down portion 40 of the same length. It should be noted that while dividers 38 form external air pockets 44 where little to no air circulates, the divider configuration of inhaler device 1 does not create any dead air pockets inside capsule recess 9 that could create unwanted turbulence and or allow some of the contents of the capsule to collect.

Referring to FIG. 9, an enlarged portion of FIG. 8 is provided and shows further dimensions of device 1. As shown, the necked down region 40 of inlet 10 has a width W. In some embodiments, width W is nominally 1.07 mm with a tolerance of plus or minus 0.05 mm (i.e. is between 1.02 and 1.12 mm, inclusive), as shown. In some embodiments, width W is about 1.10 mm. In some embodiments, width W is between 0.97 and 1.17 mm, inclusive. In some embodiments, width W is less than 1.17 mm, less than 1.10 mm, less than 1.07 mm or less than 0.97 mm. In some embodiments, each inlet may have a width W no greater than 1.15 mm. In some embodiments, capsule recess 9 has a diameter of about 19.00 mm.

The curved portion 42 located at the distal end of divider 38 may have a constant outer radius of R, as shown. In some embodiments, radius R is about 1.6 mm.

Referring again to FIG. 5, each inlet in this exemplary embodiment has the same height H as shown. In this embodiment, both the larger outer inlet portion 10 and the necked down inner inlet portion 40 have the same height H. In some embodiments, height H is no greater than the height of the recess 9 (i.e. the upper portion of recess 9 defined by outer surface 36, where the capsule spins.) In this exemplary embodiment, inlet 10 has a height H of about 5.50 mm. In this embodiment, inlet 10 has a necked down portion 40 that has a constant transverse cross-section that is rectangular. The height H of this rectangular cross-section is larger than its width W. Specifically, in this embodiment the height H of the cross-section is about 5.50 mm and the width W is about 1.07 mm, forming a cross-sectional area of about 5.89 mm². In this embodiment, the rectangular cross-sectional area remains constant (within manufacturing tolerances) along length L (shown in FIG. 8.) In some embodiments, the internal duct 12 of mouthpiece/outlet 3 has an inside diameter of about 11.00 mm.

In some embodiments of inhaler device 1, plastic mold tolerances are more tightly controlled to limit an internal bypass gap between inhaler body 2 and mouthpiece flange 4 to 0.1 mm. In some prior art devices, the internal bypass gap is 0.2 mm.

Inhaler devices with many of the features of device 1 are already known. Moreover, variations of these devices have been developed and are currently in the market. However, much analysis and experimentation has been done by the applicants to determine specific combinations of device parameters that lead to high emitted drug doses, particularly with new drug formulations being developed. Choosing an airflow resistance is one part of this device development. Prior art devices range in resistance from 0.013 to 0.185 cm H₂O^{0.5}/LPM. Advantages for having a relatively high resistance include greater powder dispersion potential. In particular, higher resistance in some inhalers will increase air velocity in the capsule chamber at a given pressure drop. This provides more energy for particle deagglomeration by: 1) increased capsule rotational velocity for improved evacuation of dose and deagglomeration due to shear through the capsule pierced holes; 2) increased turbulence in the capsule chamber for improved deagglomeration of the dose; and 3) increased particle velocity/frequency of particle impaction in the capsule chamber for improved deagglomeration of the dose. Users of dry powder inhalers (DPIs) can generate higher pressure drops when the DPI has a higher airflow resistance. This results in greater air velocities in the DPI. Maximum inspiratory effort does not seem to be affected by asthma severity. Flowrate is less sensitive to variations in inspiratory effort (pressure drop) when there is higher resistance, which in turn leads to lower variability in delivered dose. This follows the relationship Q=√P/R. For a given inspiratory effort (P), higher resistance (R) results in lower flow rate (Q), and lower flow rate results in lower exit velocity (V), which reduces the probability of oropharyngeal impaction since the probability of impaction is proportional to V*D². This lower flowrate also fills the lungs more slowly, allowing for a longer duration of inhalation, helping to ensure that the drug capsule will be fully evacuated. Higher airflow resistance also promotes opening of the throat and upper airways.

Disadvantages of a higher airflow resistance include lower exit velocities, which may increase device retention of fine particles in the mouthpiece. However, lactose-based formulations which include larger carrier particles can have a scouring effect on the mouthpiece walls and can reduce this effect. Higher resistance can also increase the influence of casework leaks such as those coming through an internal bypass gap mentioned earlier. A high resistance DPI may also be perceived as slightly less comfortable for patients to use.

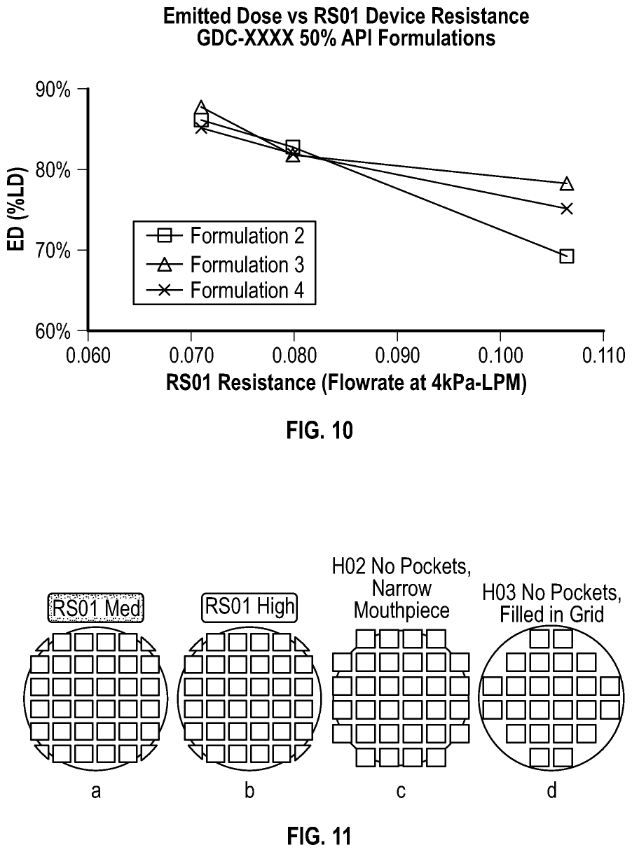

In light of the above considerations, in some embodiments, inhaler device 1 is configured to operate at a resistance of 0.128 cm H₂O^{0.5}/LPM (equivalent to a flow rate of 50 LPM at 4 kPa.) in accordance with aspects of the present disclosure. However, DPI resistance is only one important factor to consider during DPI design. The airflow interactions with the powder can also have a very significant effect on DPI performance, and can vary independently of airflow resistance. For example, as shown in FIG. 10, testing of high drug load (e.g. 50% API) formulations indicated that emitted dose decreased with increasing device resistance.

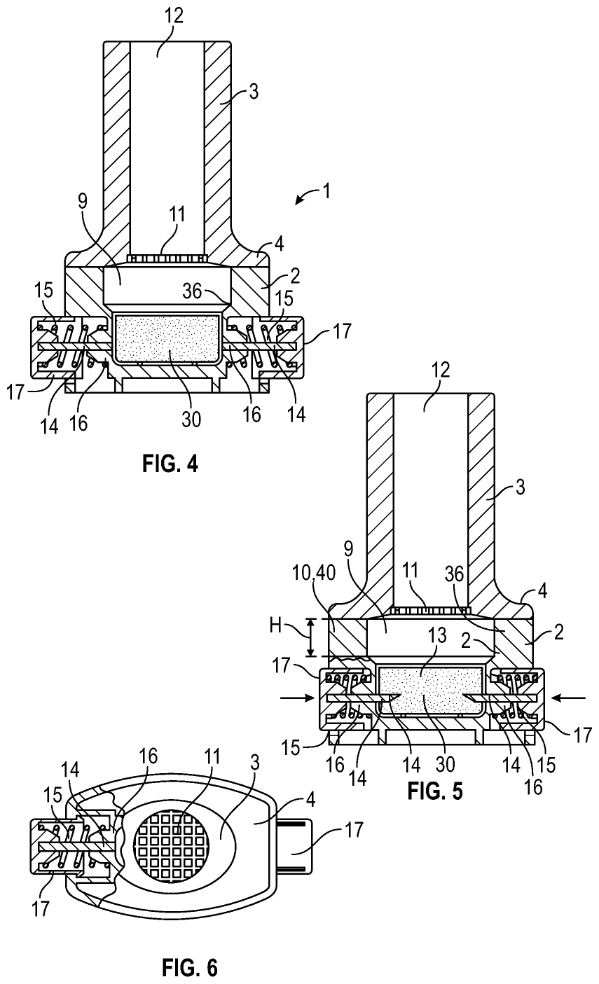

Other device parameters that can have a significant impact on device performance include the height, width, length and radius of air inlets, the existence of air pockets in the inlets and/or capsule chamber, diverging inlets, the length and diameter of the mouthpiece, and parameters associated with the grid between the capsule chamber and the mouthpiece (such as grid 11 shown in FIGS. 4-7.)

Referring to FIG. 11, some of the variations of grid filling and mouthpiece diameters that have been explored by applicants are depicted. Panel a) of FIG. 11 depicts a baseline medium resistance device found in the prior art. It has a mouthpiece ID of 10.9 mm and a grid open area of 32.2 mm². Panel b) of FIG. 11 depicts a baseline high resistance device found in the prior art. It also has a mouthpiece ID of 10.9 mm and a grid open area of 32.2 mm². Panel c) of FIG. 11 depicts a new device having a mouthpiece ID of 9.5 mm and a grid open area of 25.4 mm². This design is intended to improve swirl acceleration in the capsule chamber by increasing velocities through the mouthpiece. The ID of 9.5 mm was chosen to produce the same mouthpiece velocities as the RS01 Med device in panel a). Panel d) of FIG. 11 depicts another new device having a mouthpiece ID of 10.9 mm and a grid open area of 25.4 mm². It has a similar grid as the devices in panels a-c) but with some of the periphery of the grid filled in. Its design is intended to improve swirl acceleration in the capsule chamber by increasing velocities through the mouthpiece grid. In some embodiments of device 1, the mouthpiece ID and grid open area remain the same as the prior art devices depicted in panels a) and b).

As can be appreciated from the above discussions, there are many DPI parameters that are interrelated. When attempting to optimize one parameter, other parameters are often adversely affected. Accordingly, it is not a trivial matter to arrive at a combination of device parameters that will provide advantages such as higher emitted drug dose. Moreover, a set of device parameters that works well with one particular drug formulation may not work well with another formulation. Applicants of the present application have therefore conducted significant computational fluid dynamics (CFD) and other analyses, and have explored various permutations of device parameters to arrive at the inventive devices, formulations and drug/device combinations provided herein.

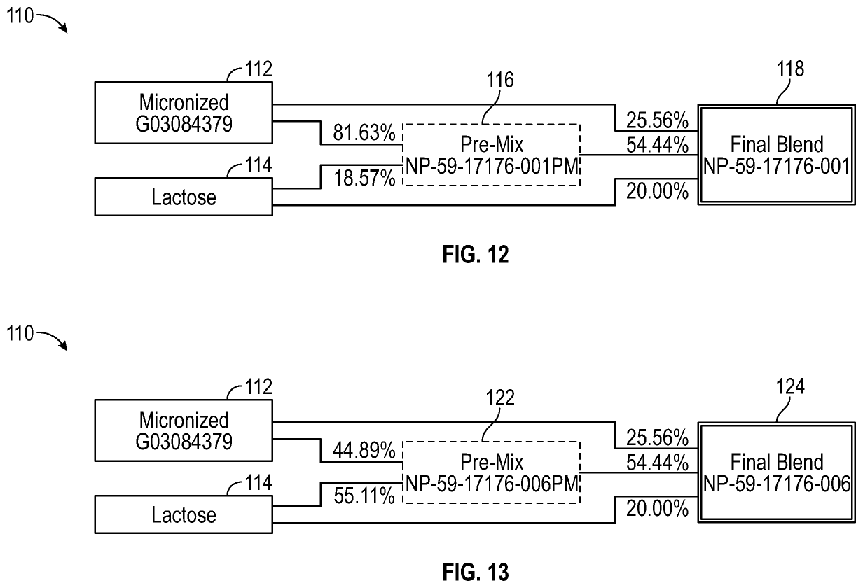

Referring to FIGS. 12-14 and according to aspects of the present disclosure, dry powder respirable drug blends and methods of formulating them for use with the inhaler devices disclosed herein are provided. In some embodiments, the dry power respirable drug blend formulations include a small molecule drug manufactured to treat asthma. The drug may include micronized crystal particles having a median size of 2 to 4 microns. In some embodiments, the drug is hydrophilic. In some embodiments, the drug is considered to be a channel hydrate. The micronized crystal particles may be blended with a lactose excipient. In some embodiments, a percentage of drug weight in the formulation is more than 10%.

In prior art drug formulations for dry powder inhalers, the percentage of active pharmaceutical ingredient (API) has typically been less than 5%. These low percentages are due in part to drugs tending to stick to themselves and form clumps that are not inhaled and absorbed well, and higher dosing of drugs not being needed in the past. There currently is a need to introduce higher amounts of newer drugs without requiring patients to inhale large quantities of excipient.

Referring to FIG. 12, a first method 110 of formulating a first dry powder respirable drug blend is provided. In this exemplary embodiment, a micronized drug 112 is provided. In some variations of method 110, micronized drug 112 is formed by first synthesizing drug molecules into crystals. The drug crystals may then be micronized, such as by using a jet mill process. A course lactose excipient 114 is also provided. A pre-mix 116 is formed by blending the micronized drug 112 and the course lactose excipient 114. In this embodiment, pre-mix 116 comprises 81.62% micronized drug 112 and 18.57% lactose 114. A final blend 118 is then formed by blending pre-mix 116 with the micronized drug 112 and lactose 114. In this embodiment, final blend 118 comprises 25.56% micronized drug 112, 54.44% pre-mix 116 and 20.00% lactose 114. With this first manufacturing scheme, final blend 118 comprises 70% micronized drug 112 or active pharmaceutical ingredient (API) and 30% lactose excipient 114.

Referring to FIG. 13, a second method 110 of formulating a second dry powder respirable drug blend is provided. In this exemplary embodiment, a micronized drug 112 and a course lactose excipient 114 are provided as previously described. A pre-mix 122 is formed by blending the micronized drug 112 and the course lactose excipient 114. In this embodiment, pre-mix 122 comprises 44.89% micronized drug 112 and 55.11% lactose 114. A final blend 124 is then formed by blending pre-mix 122 with the micronized drug 112 and lactose 114. In this embodiment, final blend 124 comprises 25.56% micronized drug 112, 54.44% pre-mix 122 and 20.00% course lactose 114. With this second manufacturing scheme, final blend 124 comprises 50% micronized drug 112 or active pharmaceutical ingredient (API) and 50% lactose excipient 114.

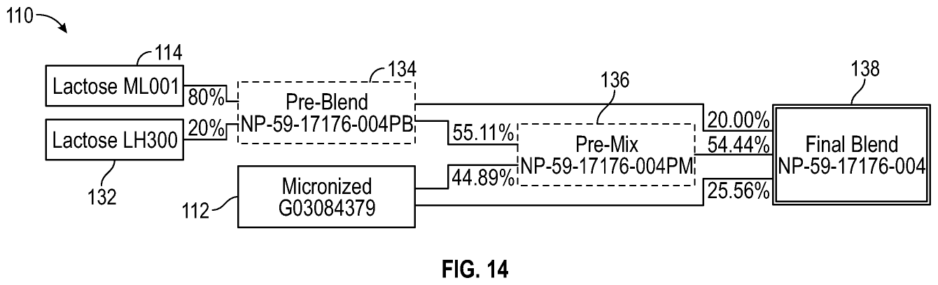

Referring to FIG. 14, a third method 110 of formulating a third dry powder respirable drug blend is provided. In this exemplary embodiment, a micronized drug 112 and a course lactose excipient 114 are provided as previously described, along with a fine lactone excipient 132. A pre-blend 134 is formed by blending the course lactose excipient 114 with the fine lactose excipient 132. In this embodiment, pre-blend 134 comprises 80% course lactose 114 and 20% fine lactose 132. A pre-mix 136 is formed by blending the micronized drug 112 and the pre-blend 134. In this embodiment, pre-mix 136 comprises 44.89% micronized drug 112 and 55.11% pre-blend 134. A final blend 138 is then formed by blending pre-mix 136 with the micronized drug 112 and pre-blend 134. In this embodiment, final blend 138 comprises 25.56% micronized drug 112, 54.44% pre-mix 136 and 20.00% pre-blend 134. With this third manufacturing scheme, final blend 138 comprises 50% micronized drug 112 or active pharmaceutical ingredient (API) and 50% lactose excipient (40% course lactose 114 and 10% fine lactose 132.) In some tests performed to date, the coarse lactose excipient 114 used was Respitose® ML001 or Respitose® SV003 and the fine lactose excipient 132 was LH300, all manufactured by DFE Pharma headquartered in Goch Germany.

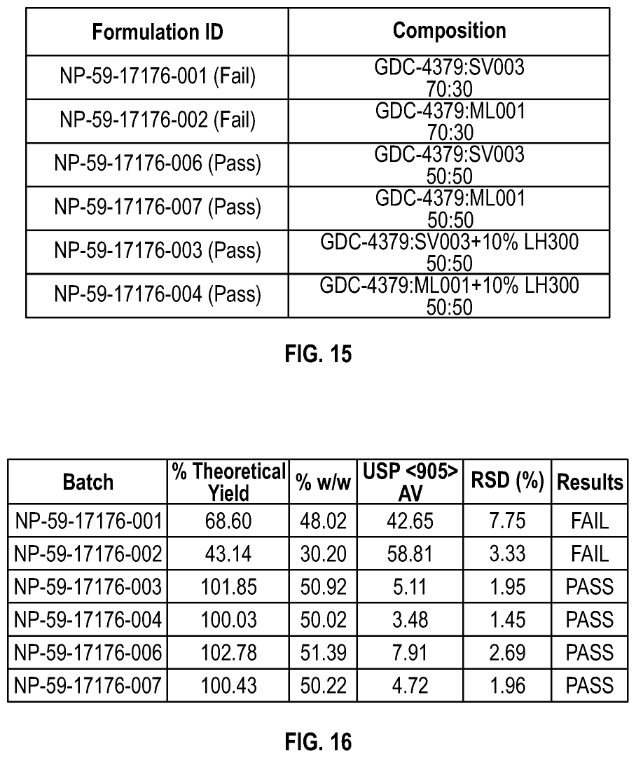

Referring to FIGS. 15 and 16, each of the three formulations described above have been manufactured and analyzed for blend homogeneity using United States Pharmacopeia (USP) <905> Uniformity of Dosage Units. The compositions are summarized in FIG. 15 and the results of the testing are shown in FIG. 16. As shown in FIG. 16, the blend content uniformities (BCUs) for both 001 and 002 formulations did not meet applicant's specification for BCU. The formulations appeared to have segregated due to the high drug load. The 50% drug load formulations with SV003 and ML001 passed the specification for BCU. These data suggest that homogenous formulation with a 50% drug load was achieved with both SV003 and ML001.

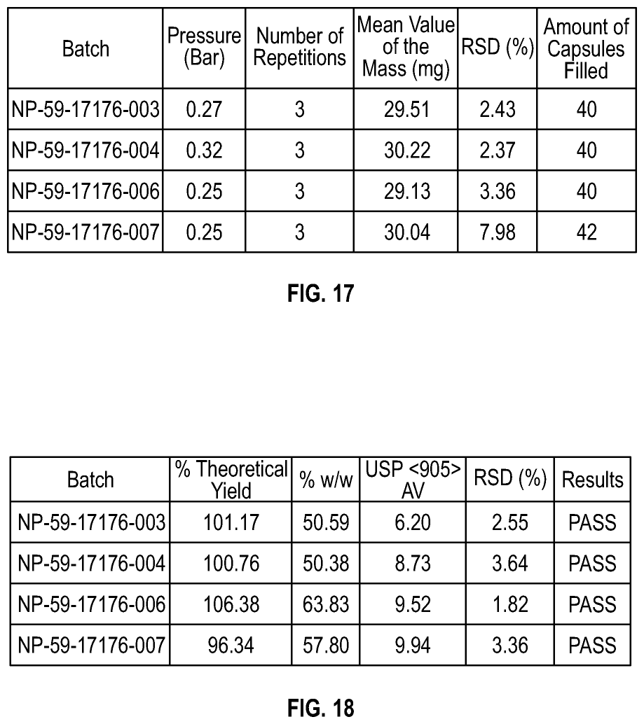

Referring to FIGS. 17 and 18, all formulations were filled into capsules using a Harro Hoffliger OmniDose TT filling system. A target fill weight of 30 mg was used with a +/−5% range. For the 50:50 drug to excipient blends this equates to 15 mg of drug per capsule. The results from these filling trials is shown in FIGS. 17 and 18. The relative standard deviation (RSD) of the capsule fill mass of formulations 003, 004 and 006 was less than 5%. In contrast, the RSD of the capsule fill mass of formulation 007 was less than 8%. These data suggest good control in the filling of these formulations using an Omnidose TT. Moreover, the capsule content uniformity of all formulations met USP<905> criteria and further demonstrated the control of the capsule filling process.

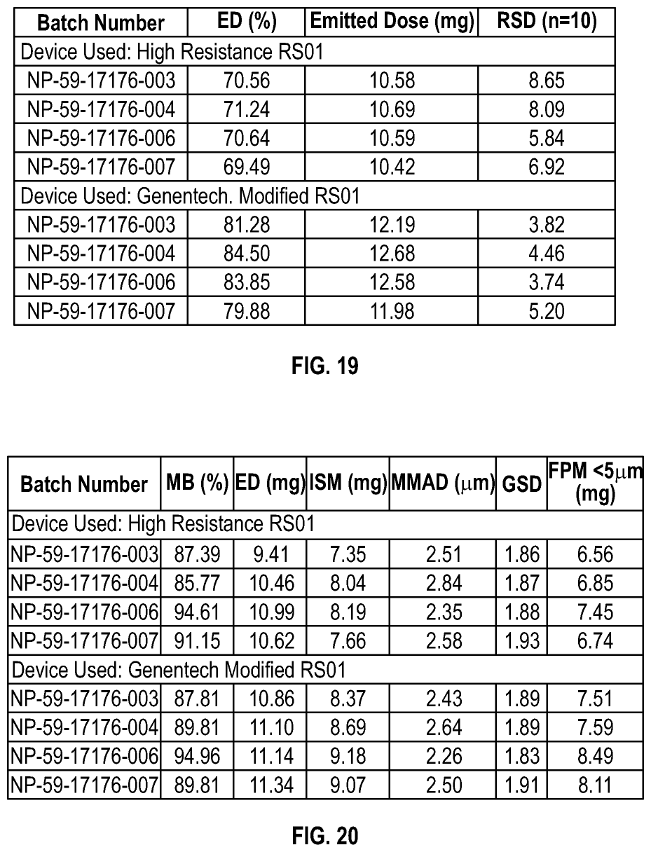

Referring to FIG. 19, testing was then performed using the filled capsules described above, both with a prior art inhaler device and with an inhaler device 1 constructed according to aspects of the present disclosure as previously described herein (also referred to herein as the GNE-RS01 device.) The prior art inhaler device used in the testing was a High Resistance Model RS01 manufactured by the Plastiape Group located in Lombardy Italy. The emitted dose as determined from single actuation content measurements using the prior art device and inhaler device 1 is shown in FIG. 19. A Dosage Unit Sampling Apparatus (DUSA) was used to perform the testing. The high-resistance RS01 testing resulted in an average emitted dose of 10 mg for all formulation tested and therefore approximately 70% emitted fraction. The uniformity of the emitted dose was well within 15%. The use of the improved inhaler device 1 resulted in an approximately 10-14% increase in the emitted fraction. The average emitted dose of all formulations was 12 mg and the uniformity of the emitted dose was well within 15%.

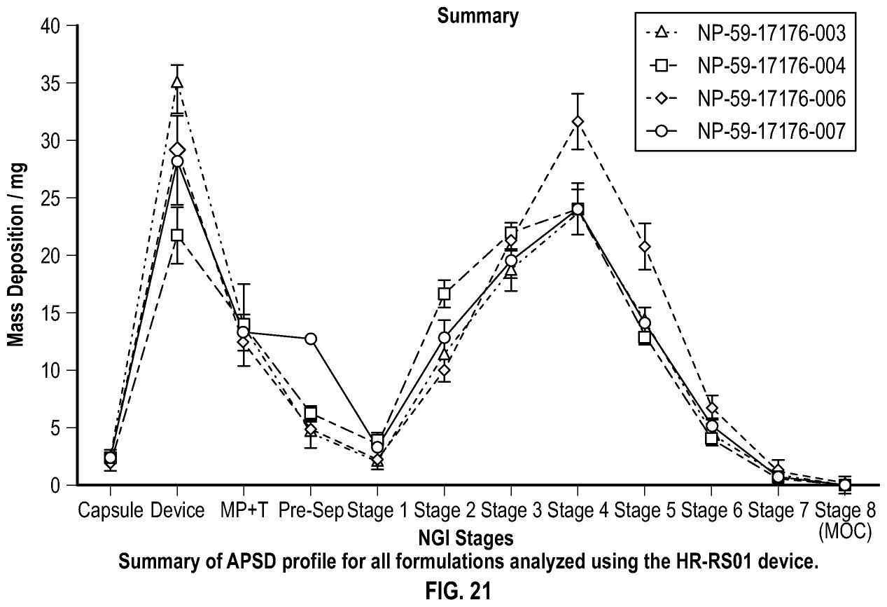

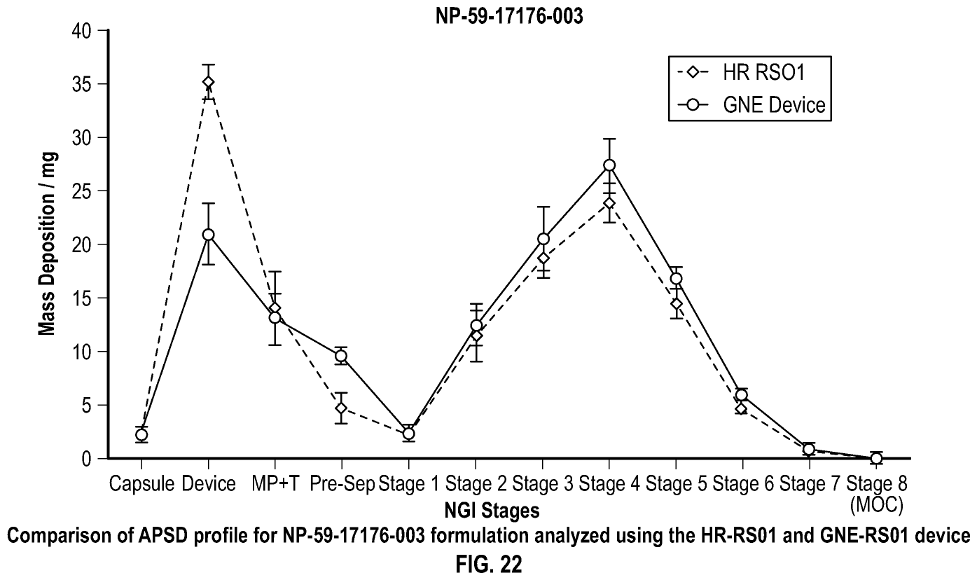

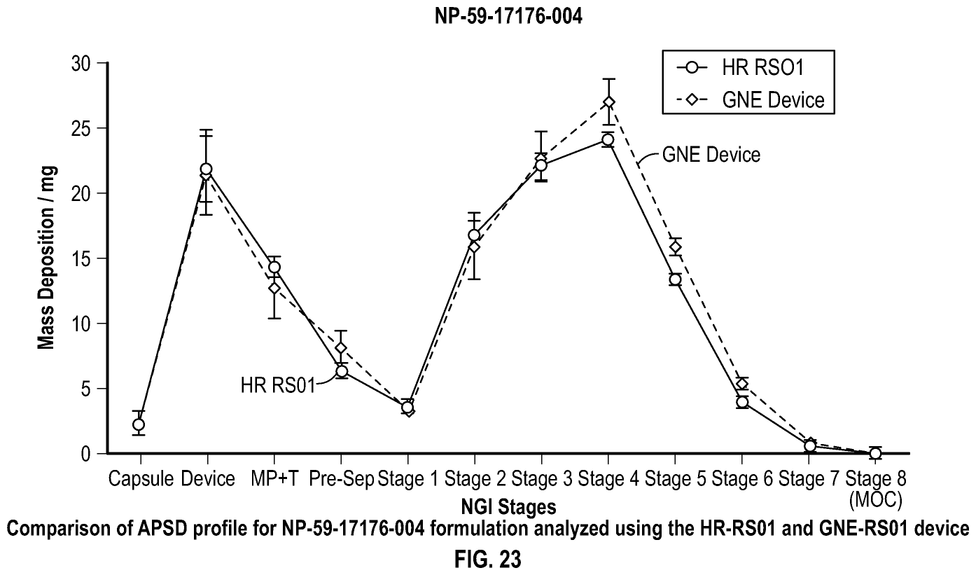

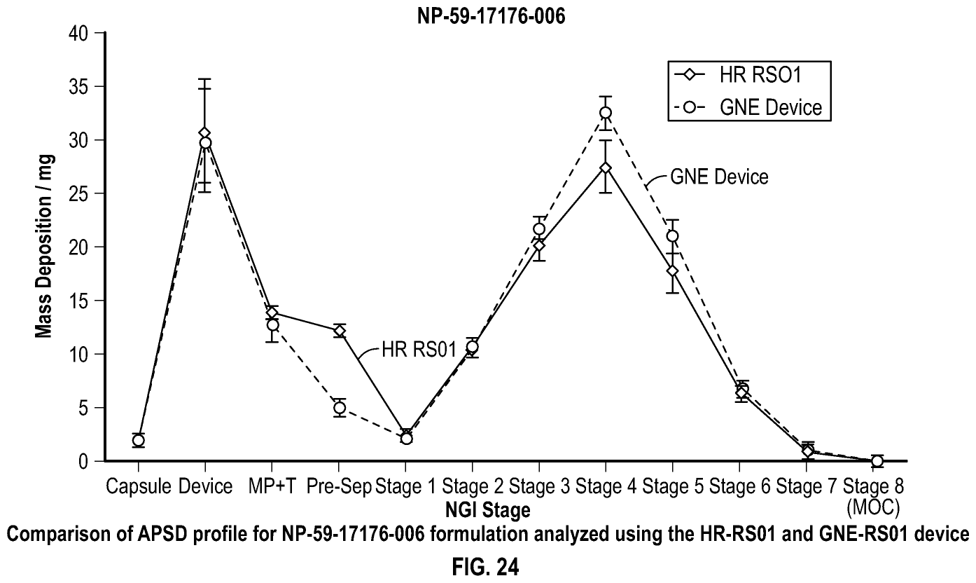

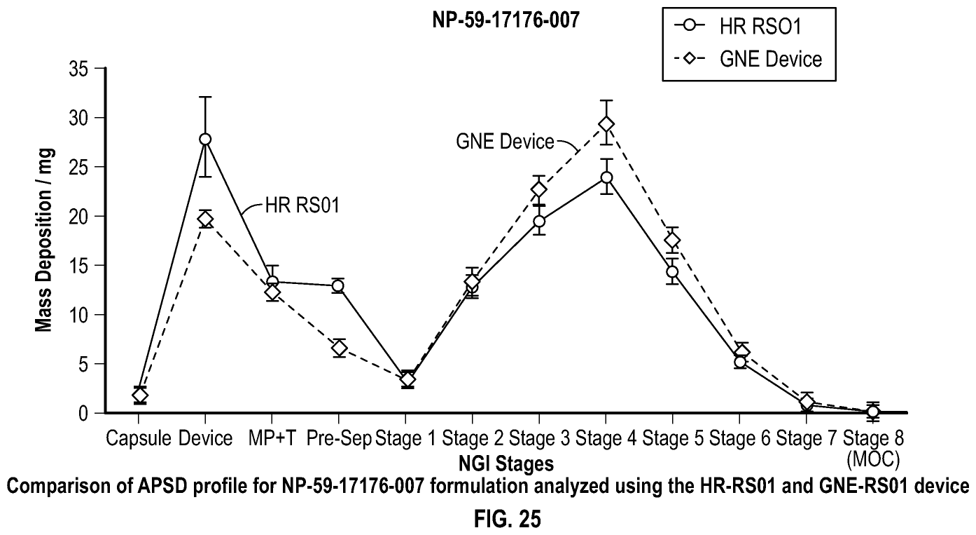

Referring to FIG. 20, Aerodynamic Particle Size Distribution (APSD) testing was performed using a Next Generation Impactor (NGI). The analysis was first carried out using the prior art RS01 device at 60 L/min. This analysis was then replicated using the improved device 1 for comparison. The flowrate for these tests were carried out at a pressure drop of 4 kPa, resulting in a 50 L/min. flowrate for the improved device 1. These results are shown in FIG. 20, along with graphs depicting the stage-by-stage deposition differences provided by FIGS. 21-25.

The introduction of fines into the formulation lactose blend results in a noticeable decrease of deposition within the pre-separator as shown in FIG. 21. The stage deposition profile for all formulations are quite similar with an exception of formulation 004, which shows increased deposition on stages 2 and 3 and formulation 006, which shows increased deposition on stages 4, 5 and 6.

With the use of the improved device 1, an increase in emitted dose shows up mostly as increased deposition on the lower stages, post stage 2, compared to that of the performance profile using the High Resistance RS01 device.

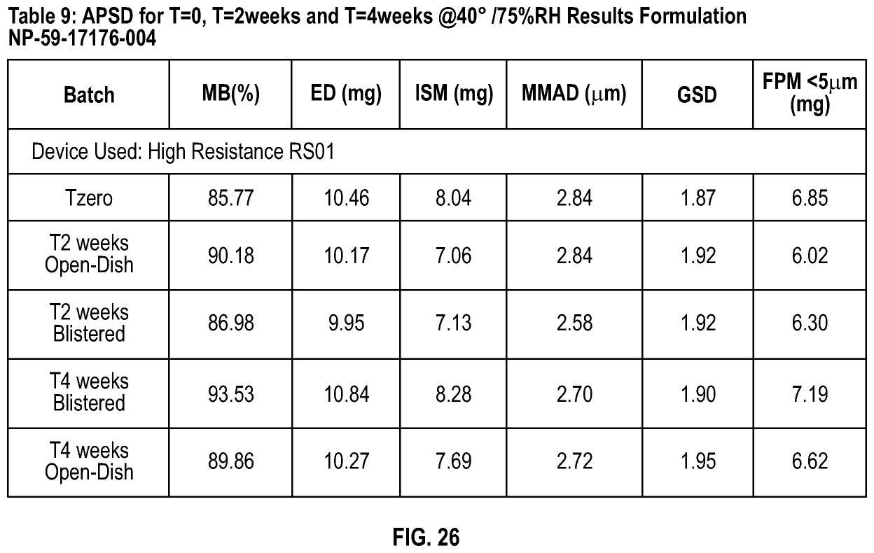

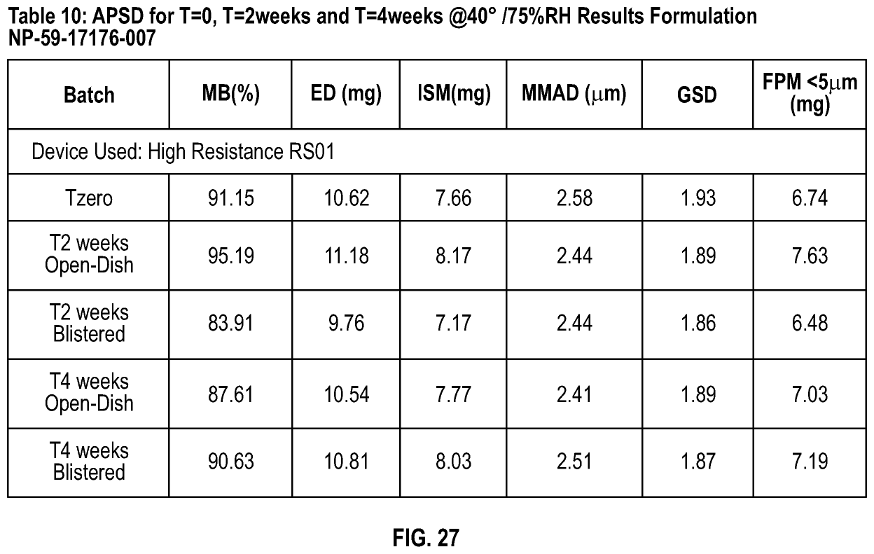

Referring to FIGS. 26 and 27, summaries of preliminary drug blend stability tests are provided. After being placed under set environmental conditions, blistered and open dish, for 2 weeks and 4 weeks, the two lead formulations were analyzed for their APSD performance. These results can be seen in FIGS. 26 and 27. In these figures, MB is the mass balance, and the Mass Median Aerodynamic Diameter (MMAD) is defined as the diameter at which 50% of the particles by mass are larger and 50% are smaller. These data suggest that changes within the APSD performance of both Formulation 004 and 007 were minimal over the four-week period. With regard to formulation 004 a slight increase in emitted dose and fine particle mass (FPM) <5 μm can be observed for both the blistered and open dish conditions, more so for the blistered capsules. However, a slight decrease in the FPM <5 μm can be observed for the 007 formulation between 2 and 4-week time points.

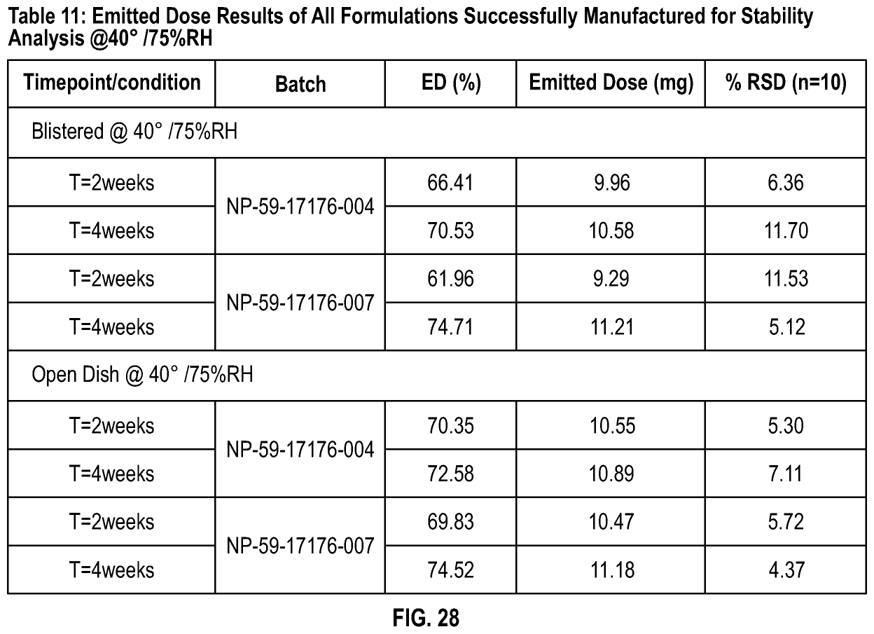

Referring to FIG. 28, further results of emitted dose stability testing by DUSA are provided. The emitted dose as determined from single actuation content measurements using the high resistance RS01 device. There is a noticeable trend in the data that shows an increase in the ED as time progresses from 2 weeks to 4 weeks. The formulation with the highest ED is the 007 when blistered and placed at 40°/75% RH for a 4-week period. This formulation shows an increase in ED by almost 5.5% from T=zero to 4 weeks.

In other embodiments, blend formulations may include a drug weight between 20 and 60%.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present disclosure.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about”, “approximately” or “generally” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the disclosure as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the disclosure as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A suction operated inhaler device, comprising a bottom inhaler body having an air inlet hole, the bottom inhaler body further defining a recess configured to hold therein a capsule containing a substance to be inhaled and a top mouthpiece communicating with the recess, the top mouthpiece having a bottom flange and being rotatably coupled to the bottom inhaler body to provide, as the top mouthpiece is manually rotated by an inhaler device user, at least two operating conditions including an open condition in which the recess for the capsule can be accessed to engage therein a new capsule or to withdraw therefrom a used capsule, and a closed use condition in which the inhaler device mouthpiece can be operated, the inhaler device further comprising at least one perforating needle associated with the inhaler body and adapted to perforate the capsule to allow a contents of the capsule to enter the capsule recess, thereby allowing an inhaling suction generated air flow passing through a first air inlet hole to mix with the contents of the capsule for inhaling the contents in the recess through the mouthpiece, wherein the first air inlet hole has a width no greater than 1.17 mm.

2. The inhaler device of claim 1, wherein the device comprises a second air inlet hole configured to cooperate with the first air inlet hole to allow air into the capsule recess to mix with the contents of the capsule for inhaling the contents in the recess through the mouthpiece, wherein the second air inlet hole has a width no greater than 1.17 mm.

3. The inhaler device of claim 2, wherein the first air inlet hole and the second air inlet hole each have a constant transverse cross-section along a predetermined length of the air inlet hole.

4. The inhaler device of claim 3, wherein the predetermined length of the air inlet hole is about 4.50 mm.

5. The inhaler of claim 3, wherein the constant transverse cross-sections are rectangular.

6. The inhaler of claim 5, wherein each rectangular cross-section has a height that is greater than a width of the cross-section.

7. The inhaler of claim 6, wherein the height of each rectangular cross-section is about 5.50 mm.

8. The inhaler of claim 7, wherein the width of each rectangular cross-section is between about 0.97 mm and about 1.17 mm, inclusive.

9. The inhaler of claim 7, wherein the width of each rectangular cross-section is between about 1.02 mm and about 1.12 mm, inclusive.

10. The inhaler of claim 1, wherein the first air inlet hole has an outwardly facing radius of about 1.60 mm.

11. The inhaler of claim 1, wherein the capsule recess has an outer wall with a constant diameter, the outer wall being continuous with no air pockets therein.

12. The inhaler of claim 11, wherein the diameter of the capsule recess is about 19.00 mm.

13. The inhaler of claim 1, wherein the mouthpiece has an inside diameter of about 11.00 mm.

14. The inhaler of claim 1, wherein the inhaler device has an internal bypass gap located between the bottom flange of the top mouthpiece and the bottom inhaler body, the internal bypass gap being no greater than about 0.1 mm.

15. The inhaler of claim 1, wherein the device has an airflow resistance of about 0.128 cmH₂O^{0.5}/LPM, which is equivalent to a flow rate of about 50 LPM at 4 kPa.

16. A dry powder inhaler device comprising:
a housing body having a cylindrically shaped recess therein, the recess having a longitudinal axis, a height along the longitudinal axis that is larger than a diameter of a capsule containing a substance to be inhaled, and a diameter transverse to the longitudinal axis that is larger than a length of the capsule, thereby allowing the capsule room to spin within the recess generally about a transverse axis of the capsule and generally about the longitudinal axis of the recess;
a pair of air inlets each fluidically connecting the recess to an aperture on an exterior surface of the housing body, each inlet having a surface that is aligned with a tangent to an outer surface of the recess, each inlet having a height no greater than the height of the recess and a width no greater than 1.17 mm; and
an outlet body coupled to the housing body and having a channel fluidically connecting the recess to an opening configured to allow a user to draw air through the opening, thereby allowing an airstream to be drawn through the air inlets, into the housing body recess where it causes the capsule to spin and eject its contents into the airstream, and through the outlet body channel and opening to deliver the substance into the user's lungs.

17. A suction operated inhaler device, comprising a bottom inhaler body having an air inlet hole, the bottom inhaler body further defining a recess configured to hold therein a capsule containing a substance to be inhaled and a top mouthpiece communicating with the recess, the top mouthpiece having a bottom flange and being rotatably coupled to the bottom inhaler body to provide, as the top mouthpiece is manually rotated by an inhaler device user, at least two operating conditions including an open condition in which the recess for the capsule can be accessed to engage therein a new capsule or to withdraw therefrom a used capsule, and a closed use condition in which the inhaler device mouthpiece can be operated, the inhaler device further comprising at least one perforating needle associated with the inhaler body and adapted to perforate the capsule to allow a contents of the capsule to enter the capsule recess, thereby allowing an inhaling suction generated air flow passing through a first air inlet hole and a second air inlet hole to mix with the contents of the capsule for inhaling the contents in the recess through the mouthpiece, wherein the first air inlet hole and the second air inlet hole each have a width no greater than 1.17 mm, wherein the first air inlet hole and the second air inlet hole each have a constant, rectangular, transverse cross-section along a predetermined length of the air inlet hole, the predetermined length being about 4.50 mm, wherein the height of each rectangular cross-section is about 5.50 mm and the width of each rectangular cross-section is between about 1.02 mm and about 1.12 mm, inclusive, wherein the first air inlet hole has an outwardly facing radius of about 1.60 mm, wherein the capsule recess has an outer wall with a constant diameter of about 19.00 mm, the outer wall being continuous with no air pockets therein, wherein the mouthpiece has an inside diameter of about 11.00 mm, wherein the inhaler device has an internal bypass gap located between the bottom flange of the top mouthpiece and the bottom inhaler body, the internal bypass gap being no greater than about 0.1 mm, and wherein the device has an airflow resistance of about 0.128 cmH₂O^{0.5}/LPM, which is equivalent to a flow rate of about 50 LPM at 4 kPa.

18. A dry powder respirable drug blend formulation comprising:
a small molecule drug manufactured to treat asthma, wherein the drug comprises micronized crystal particles having a median size of 2 to 4 microns; and
a lactose excipient, wherein a percentage of drug weight in the formulation is more than 10% and less than 70%.

19. A method of manufacturing a dry powder respirable drug blend formulation comprising the steps of:
providing a small molecule drug manufactured to treat asthma, wherein the drug comprises micronized crystal particles having a median size of 2 to 4 microns;
providing a lactose excipient; and
blending the drug with the lactose such that a percentage of drug weight in a final blend is more than 10% and less than 70%.

20. An asthma treatment product comprising:
a dry powder inhaler device configured to receive a medicine capsule; and
at least one medicine capsule containing a dry powder respirable drug blend formulation,
wherein the device comprises:
a housing body having a cylindrically shaped recess therein, the recess having a longitudinal axis, a height along the longitudinal axis that is larger than a diameter of a capsule containing a substance to be inhaled, and a diameter transverse to the longitudinal axis that is larger than a length of the capsule, thereby allowing the capsule room to spin within the recess generally about a transverse axis of the capsule and generally about the longitudinal axis of the recess;
a pair of air inlets each fluidically connecting the recess to an aperture on an exterior surface of the housing body, each inlet having a surface that is aligned with a tangent to an outer surface of the recess, each inlet having a height no greater than the height of the recess and a width no greater than 1.17 mm; and
an outlet body coupled to the housing body and having a channel fluidically connecting the recess to an opening configured to allow a user to draw air through the opening, thereby allowing an airstream to be drawn through the air inlets, into the housing body recess where it causes the capsule to spin and eject its contents into the airstream, and through the outlet body channel and opening to deliver the substance into the user's lungs,
wherein the medicine capsule comprises:
a small molecule drug manufactured to treat asthma, wherein the drug comprises micronized crystal particles having a median size of 2 to 4 microns; and
a lactose excipient, wherein a percentage of drug weight in the formulation is more than 10% and less than 70%.
